(19) United States
(12) Patent Application Publication
(10) Pub. No.: US 2009/0163908 A1
(43) Pub. Date: Jun. 25, 2009

(54) Vacuum Pre-Check for a Tissue Ablation Device

(76) Inventors: Stewart MacLean, Allston, MA (US); Doug Macbride, Woburn, MA (US); Russell Layton, JR., Acton, MA (US)

Correspondence Address: Heather Larson;Cytyc Corporation, 250 Campus Drive, Marlborough, MA, 01752, US

(21) Appl. No.: 11/960,501

(22) Filed: Dec. 19, 2007

(51) Int. Cl. A61B 18/18; A61B 19/00; A61B 5/00

(52) U.S. Cl. 606/40; 128/898; 600/561

(57) ABSTRACT

The present invention relates to a method for the removal of fluid or other liquids from a body cavity during a tissue ablation procedure. Specifically, the present invention is related to a method for determining the presence or absence of a vacuum within a device before initiating an ablation procedure.

### FIELD OF THE INVENTION

The present invention relates to a method for the removal of fluid or other liquids from a body cavity during a tissue ablation procedure. Specifically, the present invention is related to a method for determining the presence or absence of a vacuum within a device before initiating an ablation procedure.

### A BACKGROUND OF THE INVENTION

Ablation of the interior lining of a body organ is a procedure which involves heating the organ lining to temperatures which destroy the cells of the lining or coagulate tissue proteins for hemostasis. Such a procedure may be performed as a treatment to one of many conditions, such as chronic bleeding of the endometrial layer of the uterus or abnormalities of the mucosal layer of the gallbladder. Existing methods for effecting ablation include circulation of heated fluid inside the organ (either directly or inside a balloon), laser treatment of the organ lining, and resistive heating using application of radio-frequency (RF) energy to the tissue to be ablated.

Techniques using RF energy provide an RF electrical signal to one or more electrodes in contact with the subject organ tissue. Electrical current flows from the electrodes and into the organ tissue. The current flow resistively heats the surrounding tissue. Eventually, the heating process destroys the cells surrounding the electrodes and thereby effectuates ablation.

Before the start of power delivery, blood and saline solution may surround, the electrodes. As RF energy is applied to the subject organ tissue, cells surrounding the electrodes are destroyed resulting in additional blood and saline solution surrounding the electrodes. These conductive liquids act to decrease the electrode impedance. These liquids may be suctioned away during the ablation process. Absent these conductive liquids, the electrode impedance will increase with the destruction of the surrounding cells. Depending upon the specific electrode configuration, the impedance characteristics may change from as little as a fraction of an ohm to well over 200 ohms during the course of an ablation procedure. Therefore, it is imperative there exists a proper vacuum system for the removal of blood, saline, and other fluids during the ablation process.

### SUMMARY OF THE INVENTION

This invention generally relates to a vacuum system to remove fluid or other liquids from a body cavity during a tissue ablation procedure. Specifically, the present invention is related to a method for determining the presence or absence of a vacuum created by a medical device before initiating an ablation procedure.

In one embodiment of the present invention, a method of ablating and/or coagulating tissue, comprising, in the following order, the steps of: (a) providing an ablation device including an electrode array carried by an elongate tubular member, the electrode array including a fluid permeable member; (b) positioning the electrode array in contact with tissue to be ablated; (c) applying negative pressure across the fluid permeable member such that a vacuum is formed; (d) determining whether appropriate level of vacuum is formed before proceeding with ablating and/or coagulating tissue; and (e) delivering RF energy through the array to the tissue to cause the tissue to dehydrate; wherein applying suction through the fluid permeable elastic member in step (c) causes moisture generated during the dehydration of step (e) to pass into the fluid permeable member, is provided.

### BRIEF DESCRIPTION OF THE DRAWINGS

FIG. 1 is a schematic representation of the order of operation of the method of the present invention.

### DETAILED DESCRIPTION OF THE INVENTION

The invention described in this application is an aspect of a larger set of inventions described in the following patents and patent applications which are commonly owned by the assignee of the present invention, and are hereby incorporated by reference: U.S. Pat. Nos. 5,769,880, 6,508,815, 6,663,626, 6,813,520, US 2005-0085880, and US 2005-0267468.

The present invention describes a method for determining the status of a vacuum within a system to remove fluid or other liquids from a body cavity during a tissue ablation procedure. Specifically, the present invention relates to a method for an automated pre-check of vacuum pressure levels before the initiation of a Global Endometrial Ablation (GEA) procedure.

An apparatus and method for use in performing ablation or coagulation of organs and other tissue includes an electrode carrying member which is substantially absorbent and/or permeable to moisture and gases such as steam and conformable to the body cavity. Suctioning means may additionally be positioned within the electrode carrying member to aide the removal of moisture, and/or gas and/or liquid, present or generated during the ablation procedure. An array of electrodes is mounted to the surface of the electrode carrying member and arranged to produce ablation to a predetermined depth. The electrodes may be provided with means for variably controlling ablation depth by changing the electrode density or center to center spacing.

Following placement of the ablation device into contact with the tissue to be ablated, an RF generator is used to deliver RF energy to the electrodes and to thereby induce current flow from the electrodes to tissue to be ablated. As the current heats the tissue, moisture (such as steam or liquid) leaves the tissue causing the tissue to dehydrate. The moisture permeability and/or absorbency of the electrode carrying member allows moisture to leave the ablation site so as to prevent the moisture from providing a path of conductivity for the current. A currently available ablation system, the NovaSure® MTS System (Hologic, Marlborough. Mass.) utilizes a vacuum to remove fluid and water vapor from a patient's uterine cavity during the ablation procedure. The vacuum provides moisture control to assist with maintaining proper application of energy to the target tissue, as well as helping to maintain constant contact between the electrode array and the endometrial lining of the uterus.

Currently, in the NovaSure® MTS System, a vacuum is applied to the target tissue at the time of application of RF energy to the electrodes. If there is insufficient vacuum, the application of RF energy will terminate and the system will shut down. The operator must then determine the source of the vacuum error and then restart the procedure again.

The method of the present invention describes a method to determine whether or not there is an appropriate vacuum level being pulled by an ablation device before the application of energy to the electrode array begins. More importantly, the method of the present invention describes a system which incorporates a vacuum pre-check step which would help with determining the source of vacuum leaks before the application of RF energy to the target tissues.

In particular, the order of operation according the present invention is comprised generally the steps of (a) providing an ablation device including an electrode array carried by an elongate tubular member, the electrode array including a fluid permeable elastic member having insulating regions and conductive regions thereon; (b) positioning the electrode array in contact with tissue to be ablated; (c) applying negative pressure across the fluid permeable elastic member such that a vacuum is formed; (d) determining whether appropriate level of vacuum is formed before proceeding with ablating and/or coagulating tissue; and (e) delivering RF energy through the array to the tissue to cause the tissue to dehydrate; wherein applying suction through the fluid permeable elastic member in step (c) causes moisture generated during the dehydration of step (e) to pass into the fluid permeable elastic member, away from tissue and into the tubular member the suction substantially eliminating liquid surrounding the electrodes during ablation.

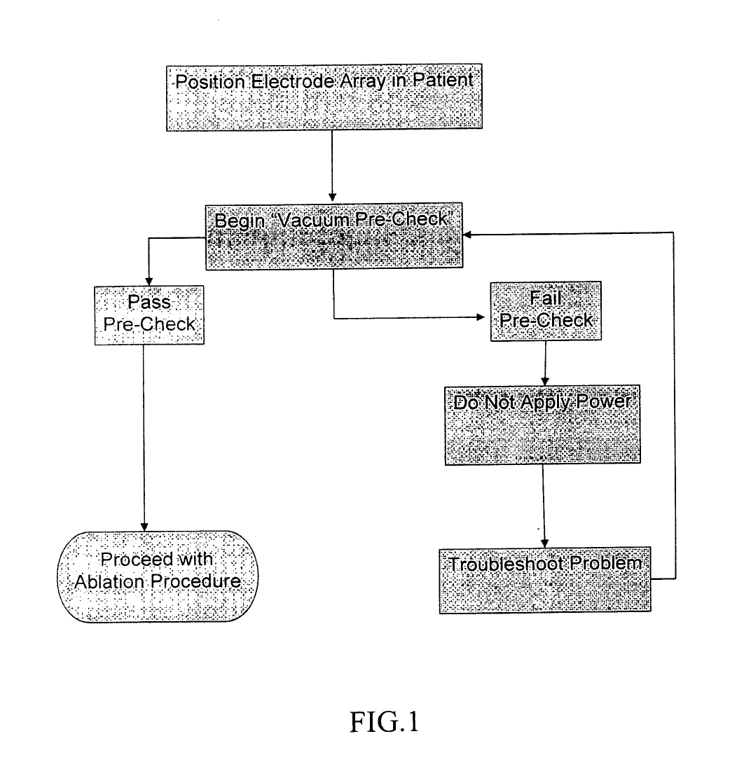

Referring to FIG. 1, the order of operation according the present invention is comprised generally the steps of positioning the electrode array in a patient, forming a vacuum, and beginning a vacuum pre-check to determine whether appropriate level of vacuum is formed before proceeding with an ablation and/or coagulation procedure. If the vacuum pre-check fails, energy is not applied to the target tissue until the error is corrected. If the vacuum pre-check passes, the ablation procedure may continue.

More specifically, an ablation system is designed to verify that there is an appropriate vacuum level present before power application to the electrode array begins. This means that when an operator, such as a surgeon, is about to being an ablation procedure, he/she would initiate a “vacuum pre-check” step which interrogates the system to determine if an appropriate vacuum is being applied. The appropriate vacuum is a predetermined value or a range of values based upon the type of tissue or organ being ablated. The predetermined vacuum value (or range of values) may be compared to the current operating level of the vacuum (detected by an ablation device). After comparing these values, a determination may be made about whether or not the current vacuum operation level is appropriate. After making such a determination, a signal, such as a “pass” or “fail” signal will be communicated to a user or operator. Once the vacuum level has been confirmed to be within an acceptable range, the device operator may apply energy through the electrode array to ablate the target tissue or organ.

If the “vacuum pre-check” step is not able to confirm the presence of a proper vacuum, the system will prevent any power from being applied to the electrode array and a “fail” signal may be sent to the user. When the “vacuum pre-check” fails or is unable to confirm the presence of a proper vacuum, then troubleshooting procedures may also be performed to try and detect and correct the source of the vacuum error or failure. In some methods of operation or use, the “vacuum pre-check” step may be performed multiple times (i.e., may be an iterative process). The “vacuum pre-check” may continue to detect current levels of operation and compare that value to a predetermined vacuum value (or range of values) until the vacuum level has been confirmed to be within the acceptable range. This “vacuum pre-check” allows for the determination of optimal operating conditions before the ablation procedure can begin. Specific embodiments of methods of the present invention are described below

In one embodiment of the present invention, a method of ablating and/or coagulating tissue, comprising, in the following order, the steps of: (a) providing an ablation device including an expandable electrode array carried by an elongate tubular member and a pair of elongate flexures wherein each flexure includes at least one opening, the electrode array including a fluid permeable elastic member having insulating regions and conductive regions thereon; (b) positioning the electrode array into an organ and into contact with tissue to be ablated and moving the array to an expanded condition by expanding the flexures; (c) measuring the approximate length and width of the organ, selecting an ablation power corresponding to the measured length and width, (d) applying negative pressure across the fluid permeable elastic member such that a vacuum is formed; (e) determining whether appropriate level of vacuum is formed before proceeding with ablating and/or coagulating tissue; (f) delivering RF energy through the array to the tissue at approximately the selected power to cause the tissue to dehydrate, and (g) permitting moisture generated during the dehydration of step (f) to pass into the fluid permeable elastic member and away from the tissue and allowing at least a portion of the moisture to pass through the openings in the flexures is provided.

In another embodiment of the present invention, the step of measuring the approximate width of the organ includes the step of expanding the flexures to an expanded condition and deriving the approximate width of the uterus from the relative positions of the flexures in the expanded condition. In yet another embodiment of the present invention an ablation power which is proportional to the measured length times the measured width is provided.

In yet another embodiment of the present invention, a method of ablating and/or coagulating tissue, comprising, in the following order, the steps of: (a) providing an ablation device including an electrode array carried by an elongate tubular member, the electrode array including a fluid permeable elastic member having insulating regions and conductive regions thereon; (b) positioning the electrode array in contact with tissue to be ablated; (c) applying negative pressure across the fluid permeable elastic member such that a vacuum is formed; (d) determining whether appropriate level of vacuum is formed before proceeding with ablating and/or coagulating tissue; and (e) delivering RF energy through the array to the tissue to cause the tissue to dehydrate; wherein applying suction through the fluid permeable elastic member in step (c) causes moisture generated during the dehydration of step (e) to pass into the fluid permeable elastic member, away from tissue and into the tubular member the suction substantially eliminating liquid surrounding the electrodes during ablation is provided.

In still yet another embodiment of the present invention, a method of ablating and/or coagulating tissue, comprising, in the following order, the steps of: (a) providing an ablation device including an expandable electrode array carried by an elongate tubular member and a pair of elongate flexures wherein each flexure includes at least one opening, the electrode array including a fluid permeable elastic member having insulating regions and conductive regions thereon; (b) positioning the electrode array into an organ and into contact with tissue to be ablated and moving the array to an expanded condition by expanding the flexures; (c) measuring the approximate length and width of the organ, selecting an ablation power corresponding to the measured length and width, (d) applying negative pressure across the fluid permeable elastic member such that a vacuum is formed; (e) determining whether appropriate level of vacuum is formed before proceeding with ablating and/or coagulating tissue; (f) delivering RF energy through the array to the tissue at approximately the selected power to cause the tissue to dehydrate; and (g) permitting moisture generated during the dehydration of step (f) to pass into the fluid permeable elastic member and away from the tissue and allowing at least a portion of the moisture to pass through the openings in the flexures is provided.

In yet another embodiment of the present invention, a method of ablating and/or coagulating tissue, comprising, in the following order, the steps of: (a) providing an ablation device including an expandable electrode array carried by an elongate tubular member and a pair of elongate flexures wherein each flexure includes at least one opening, the electrode array including a fluid permeable elastic member having insulating regions and conductive regions thereon, wherein the array material has elasticity in a transverse direction and in a longitudinal direction and wherein the elasticity in the transverse direction is greater than the elasticity in the longitudinal direction (b) positioning the electrode array in contact with tissue to be ablated and moving the array to an expanded condition by expanding the flexures; (c) applying negative pressure across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; and (f) permitting moisture generated during the dehydration of step (e) to pass into the fluid permeable elastic member and away from the tissue and allowing at least a portion of the moisture to pass through the openings in the flexures is provided.

In still yet another embodiment of the present invention, a method of ablating and/or coagulating tissue, comprising, in the following order, the steps of: (a) providing an ablation device including an electrode array carried by an elongate tubular member, the electrode array including a fluid permeable elastic member having insulating regions and conductive regions thereon, wherein the fluid permeable elastic member includes metallized fabric; (b) positioning the electrode array in contact with tissue to be ablated; (c) applying negative pressure across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; and (f) permitting moisture generated during the dehydration of step (e) to pass into the fluid permeable elastic member, away from tissue and into the tubular member is provided.

In another embodiment of the present invention, a method of ablating and/or coagulating tissue, comprising, in the following order, the steps of: (a) providing an ablation device including an electrode array carried by an elongate tubular member, the electrode array including a fluid permeable elastic member having insulating regions and conductive regions thereon, wherein the array material has elasticity in a transverse direction and in a longitudinal direction and wherein the elasticity in the transverse direction is greater than the elasticity in the longitudinal direction; (b) positioning the electrode array in contact with tissue to be ablated; (c) applying negative pressure across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; and (f) permitting moisture generated during the dehydration of step (e) to pass into the fluid permeable elastic member, away from tissue and into the tubular member is provided.

In yet another embodiment of the present invention, a method of ablating and/or coagulating tissue, comprising, in the following order, the steps of: (a) providing an ablation device including an expandable bipolar electrode array carried by an elongate tubular member and a pair of elongate flexures wherein each flexure includes at least one opening, the electrode array comprising a fluid permeable elastic member having insulating regions and conductive regions thereon; (b) positioning the electrode array in contact with tissue to be ablated and moving the array to an expanded condition by expanding the flexures; (c) applying negative pressure across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying a vacuum through the fluid permeable elastic member in step (c) causes moisture generated during the dehydration of step (e) to pass into the fluid permeable elastic member and away from the tissue and allowing at least a portion of the moisture to pass through the openings in the flexures is provided.

In still yet another embodiment of the present invention, a method of ablating and/or coagulating tissue, comprising, in the following order, the steps of: (a) providing an ablation device including a bipolar electrode array carried by an elongate tubular member, the electrode array including a fluid permeable elastic member having insulating regions and conductive regions thereon; (b) positioning the electrode array in contact with tissue to be ablated; (c) applying negative pressure across the fluid permeable elastic member such that a vacuum is formed; (d) determining whether appropriate level of vacuum is formed before proceeding with ablating and/or coagulating tissue; (e) delivering RE energy through the array to the tissue to cause the tissue to dehydrate; wherein applying a vacuum through the fluid permeable elastic member in step (c) causes moisture generated during the dehydration of step (e) to pass into the fluid permeable elastic member, away from tissue and into the tubular member is provided.

In yet embodiment of the present invention, a method of ablating and/or coagulating tissue, comprising, in the following order, the steps of: (a) providing an ablation device including an expandable bipolar electrode array carried by an elongate tubular member, the electrode array including a fluid permeable elastic member having insulating regions and conductive regions thereon; (b) positioning the electrode array in contact with tissue to be ablated and moving the array to an expanded condition; (c) applying negative pressure through the tubular member and across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying suction to the tubular member and through the fluid permeable elastic member in step (c) causes moisture generated during the dehydration of step (e) to pass into the fluid permeable elastic member and away from the tissue, the suction drawing the moisture through the tubular member is provided.

In yet embodiment of the present invention, a method of ablating and/or coagulating tissues comprising, in the following order, the steps of: (a) providing an ablation device including an expandable electrode array carried by an elongate tubular member and a pair of elongate flexures wherein each flexure includes at least one opening, the electrode array including a fluid permeable elastic member having insulating regions and conductive regions thereon; (b) positioning the electrode array in contact with tissue to be ablated and moving the array to an expanded condition by expanding the flexures; (c) applying negative pressure through the tubular member and across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying suction through the fluid permeable elastic member in step (c) causes moisture generated during the dehydration of step (e) to pass into the fluid permeable elastic member and away from the tissue and allowing at least a portion of the moisture to pass through the openings in the flexures, the suction substantially eliminating liquid surrounding the electrodes during ablation is provided.

In yet embodiment of the present invention, a method of ablating and/or coagulating tissue, comprising, in the following order, the steps of: (a) providing an ablation device including an electrode array carried by an elongate tubular member, the electrode array including a fluid permeable elastic member having insulating regions and conductive regions thereon; (b) positioning the electrode array in contact with tissue to be ablated; (c) applying negative pressure through the tubular member and across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying suction through the fluid permeable elastic member in step (c) causes moisture generated during the dehydration of step (e) to pass into the fluid permeable elastic member, away from tissue and into the tubular member the suction substantially eliminating liquid surrounding the electrodes during ablation is provided.

In still yet another embodiment of the present invention, a method of ablating and/or coagulating tissue, comprising, in the following order, the steps of: (a) providing an ablation device including an expandable electrode array carried by an elongate tubular member, the electrode array including a fluid permeable elastic member having insulating regions and conductive regions thereon; (b) positioning the electrode array in contact with tissue to be ablated and moving the array to an expanded condition; (c) applying negative pressure through the tubular member and across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying suction to the tubular member and through the fluid permeable elastic member in step (c) causes moisture generated during the dehydration of step (e) to pass into the fluid permeable elastic member and away from the tissue, the suction drawing the moisture through the tubular member, the suction substantially eliminating liquid surrounding the electrodes during ablation is provided.

In yet another embodiment of the present invention, a method of ablating and/or coagulating tissue, comprising, in the following order, the steps of: (a) providing an ablation device including an expandable electrode array carried by an elongate tubular member and a pair of elongate flexures wherein each flexure includes at least one opening, the electrode array including a fluid permeable elastic member comprising a moisture permeable envelope having a hollow interior and having insulating regions and conductive regions thereon; (b) positioning the electrode array in contact with tissue to be ablated and moving the array to an expanded condition by expanding the flexures; (c) applying negative pressure through the tubular member and across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying a suction through the fluid permeable elastic member in step (c) causes moisture generated during the dehydration of step (e) to pass into the fluid permeable elastic member and away from the tissue and allowing at least a portion of the moisture to pass through the openings in the flexures, wherein the suction causes the moisture to pass into the hollow interior of the fluid permeable elastic member and away from the electrode array is provided.

In yet another embodiment of the present invention, method of ablating and/or coagulating tissue, comprising, in the following order, the steps of: (a) providing an ablation device including an electrode array carried by an elongate tubular member, the electrode array including a fluid permeable elastic member comprising a moisture permeable envelope having a hollow interior and having insulating regions and conductive regions thereon; (b) positioning the electrode array in contact with tissue to be ablated; (c) applying negative pressure through the tubular member and across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying suction through the fluid permeable elastic member in step (c) causes moisture generated during the dehydration of step (e) to pass into the fluid permeable elastic member, away from tissue and into the tubular member and wherein the suction causes the moisture to pass into the hollow interior of the fluid permeable elastic member and away from the electrode array is provided.

In yet another embodiment of the present invention a method of ablating and/or coagulating tissue, comprising in the following order, the steps of: (a) providing an ablation device including an expandable electrode array carried by an elongate tubular member, the electrode array including a fluid permeable elastic member comprising a moisture permeable envelope having a hollow interior and having insulating regions and conductive regions thereon; (b) positioning the electrode array in contact with tissue to be ablated and moving the array to an expanded condition; (c) applying negative pressure through the tubular member and across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; wherein applying suction to the tubular member and through the fluid permeable elastic member in step (c) causes moisture generated during the dehydration of step (e) to pass into the fluid permeable elastic member and away from the tissue, the suction drawing the moisture through the tubular member, and wherein the suction causes the moisture to pass into the hollow interior of the fluid permeable elastic member and away from the electrode array is provided.

In each of the described embodiments, the fluid permeable elastic member includes metallized fabric. The metallized fabric includes yarns of elastic material and yarns of inelastic material including, but not limited to spandex and nylon.

Exemplary values for an appropriate level of vacuum formed before proceeding with ablating and/or coagulating tissue can range from about 0.1 to about 10 inches of mercury (Hg) and will often be from about 0.7 to about 6.0 inches of Hg. In another embodiment, the appropriate level of vacuum is 0.7+/−0.2 to 6.0+/−1.0 inches of Hg.

In each of the described embodiments, the suction draws tissue into contact with the electrode carrying member. If the tissue is inside an organ, the suction at least partially collapses the organ onto the electrode carrying member. If the tissue is within a uterus, the electrode array is passed through the cervix and into the uterus. Once the electrode array is passed into the uterus the method further includes forming a seal around the elongate tubular member at the cervix.

In each of the described embodiments suction substantially prevents formation of a low-impedance liquid layer around the electrode array during ablation/coagulation using the electrode array.

In each of the described embodiments the entire bipolar electrode array maintains continuous contact with the tissue to be ablated during said ablation and/or coagulation of the tissue.

The invention can be embodied in other specific forms without departing from the spirit or essential characteristics thereof. The present embodiments are therefore to be considered in all respects as illustrative and not restrictive. The scope of the invention is indicated by the appended claims, rather than by the foregoing description, and all changes which come within the meaning and range of equivalency of the claims are therefore intended to be embraced therein.

That which is claimed:

1. A method of ablating and/or coagulating tissue, comprising, in the following order, the steps of: (a) providing an ablation device including an electrode array carried by an elongate tubular member, the electrode array including a fluid permeable elastic member having insulating regions and conductive regions thereon; (b) positioning the electrode array in contact with tissue to be ablated; (c) applying negative pressure across the fluid permeable elastic member such that a vacuum is formed; (d) determining whether appropriate level of vacuum is formed before proceeding with ablating and/or coagulating tissue; and (e) delivering RF energy through the array to the tissue to cause the tissue to dehydrate; wherein applying suction through the fluid permeable elastic member in step (c) causes moisture generated during the dehydration of step (e) to pass into the fluid permeable elastic member, away from tissue and into the tubular member the suction substantially eliminating liquid surrounding the electrodes during ablation.

2. The method of claim 1 wherein the metallized fabric includes yarns of elastic material and yarns of inelastic material.

3. The method of claim 2 wherein the metallized fabric includes yarns of spandex and nylon.

4. The method of claim 1 wherein the level of vacuum is in the range of about 1 to about 6 inches of mercury.

5. The method of claim 1, wherein the step of determining whether appropriate level of vacuum is formed includes verifying that vacuum is functioning.

6. The method of claim 1, wherein the step of determining whether appropriate level of vacuum is formed includes determining current operating level of vacuum and comparing current operating level of vacuum to a predetermined vacuum value or range of vacuum values and communicating a signal to a user based upon that comparison.

7. The method of claim 6, wherein communicating a signal to a user informs a user whether or not appropriate level of vacuum is formed.

8. The method of claim 6, wherein communicating a signal to a user informs a user whether or not ablation and/or coagulation may proceed.

9. The method of claim 1, wherein steps (c) and (d) may be performed iteratively until an appropriate level of vacuum has been determined.

10. A method of ablating and/or coagulating tissue, comprising, in the following order, the steps of: (a) providing an ablation device including an expandable electrode array carried by an elongate tubular member and a pair of elongate flexures wherein each flexure includes at least one opening, the electrode array including a fluid permeable elastic member having insulating regions and conductive regions thereon; (b) positioning the electrode array into an organ and into contact with tissue to be ablated and moving the array to an expanded condition by expanding the flexures; (c) measuring the approximate length and width of the organ, selecting an ablation power corresponding to the measured length and width, (d) applying negative pressure across the fluid permeable elastic member such that a vacuum is formed; (e) determining whether appropriate level of vacuum is formed before proceeding with ablating and/or coagulating tissue; (f) delivering RF energy through the array to the tissue at approximately the selected power to cause the tissue to dehydrate; and (g) permitting moisture generated during the dehydration of step (f) to pass into the fluid permeable elastic member and away from the tissue and allowing at least a portion of the moisture to pass through the openings in the flexures.

11. The method of claim 10 wherein the step of measuring the approximate width of the organ includes the step of expanding the flexures to an expanded condition and deriving the approximate width of the uterus from the relative positions of the flexures in the expanded condition.

12. The method of claim 11 wherein step (c) further includes selecting an ablation power which is proportional to the measured length times the measured width.

13. The method of claim 10 wherein the level of vacuum is in the range of about 1.0 to 6.0 inches of mercury.

14. A method of ablating and/or coagulating tissue, comprising, in the following order, the steps of: (a) providing an ablation device including an expandable electrode array carried by an elongate tubular member and a pair of elongate flexures wherein each flexure includes at least one opening, the electrode array including a fluid permeable elastic member having insulating regions and conductive regions thereon, wherein the array material has elasticity in a transverse direction and in a longitudinal direction and wherein the elasticity in the transverse direction is greater than the elasticity in the longitudinal direction (b) positioning the electrode array in contact with tissue to be ablated and moving the array to an expanded condition by expanding the flexures; (c) applying negative pressure across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; and (f) permitting moisture generated during the dehydration of step (e) to pass into the fluid permeable elastic member and away from the tissue and allowing at least a portion of the moisture to pass through the openings in the flexures.

15. A method of ablating and/or coagulating tissue, comprising, in the following order, the steps of: (a) providing, an ablation device including an electrode array carried by an elongate tubular member, the electrode array including a fluid permeable elastic member having insulating regions and conductive regions thereon, wherein the fluid permeable elastic member includes metallized fabric; (b) positioning the electrode array in contact with tissue to be ablated; (c) applying negative pressure across the fluid permeable elastic member such that a vacuum is formed; (d) determining whether appropriate level of vacuum is formed before proceeding with ablating and/or coagulating tissue; (e) delivering RF energy through the array to the tissue to cause the tissue to dehydrate; and (f) permitting moisture generated during the dehydration of step (e) to pass into the fluid permeable elastic member, away from tissue and into the tubular member.

16. The method of claim 15 wherein the metallized fabric includes yarns of elastic material and yarns of inelastic material.

17. The method of claim 16 wherein the metallized fabric includes yarns of spandex and nylon.

18. The method of claim 15 wherein the level of vacuum is in the range of about 1.0 to 6.0 inches of mercury.
